(19) United States
(12) Patent Application Publication
(10) Pub. No.: US 2025/0072801 A1
(43) Pub. Date: Mar. 6, 2025

(54) SYSTEM AND METHOD OF REAL TIME COGNITIVE STRESS DETECTION

(71) Applicant: HAPPY HEALTH, INC., Austin, TX (US)

(72) Inventors: Conrad W. Rosenbrock, Provo, UT (US); Shivkumar Sabesan, San Mateo, CA (US); David E. Clift-Reaves, Austin, TX (US)

(21) Appl. No.: 18/822,149

(22) Filed: Aug. 31, 2024

Related U.S. Application Data
(60) Provisional application No. 63/579,989, filed on Sep. 1, 2023.

(51) Int. Cl. A61B 5/16; A61B 5/0531

(52) U.S. Cl. CPC A61B 5/165; A61B 5/0531

(57) ABSTRACT

A computer-implemented method for detecting cognitive stress events using a wearable electronic device is disclosed. The computer-implemented method includes (i) segmenting an EDA sensor signal into rising and falling regions; (ii) segmenting rising regions and falling regions into a plurality of rise sub-regions and a plurality of fall sub-regions, respectively; (iii) fitting a transfer function to each rise sub-region of the plurality of rise sub-regions; (iv) fitting a decaying function to each fall sub-region of the plurality of fall sub-regions; (v) combining fit parameters to identify baseline rises; (vi) combining decaying fit parameters to identify baseline falls; and (vii) identifying or characterizing physiological events, based upon the baseline rises and the baseline falls, for detecting the cognitive stress events. The wearable electronic device includes at least one electrodermal activity (EDA) sensor and at least one contact area.

### CROSS-REFERENCE TO RELATED APPLICATION

This application is a nonprovisional and claims the benefit under 35 U.S.C. § 119 (e) of U.S. Provisional Patent Application No. 63/579,989, filed Sep. 1, 2023, the contents of which are incorporated herein by reference as if fully disclosed herein.

### TECHNICAL FIELD

Embodiments described herein generally relate to a system and a method for real-time cognitive stress detection using a wearable electronic device including multiple sensors, and in particular, real-time cognitive stress detection using a baseline characterizing technique in presence of confounding environmental factors.

### BACKGROUND

Wearable electronic devices such as smart watches, fitness trackers, glucose trackers, etc., include sensors for continuous monitoring of physiological and/or biological signals of a user when a wearable electronic device is worn by the user. The sensors included in the wearable electronic device monitor health-related signals such as step count, heart rate, blood glucose level, blood pressure, respiratory rate, galvanic skin conductance (or electrodermal activity (EDA)), and many others. Sensor data, based upon the monitored signals, helps the user to manage health status and empower the user in self-management of chronic disease conditions for diabetes, obesity, and cardiovascular disease by incorporating certain lifestyle changes.

EDA is correlated with cognitive stress due to autonomic nervous system (ANS) activity. The cognitive stress level is measured or accessed from raw EDA data in a laboratory like environment where a subject person is remarkably still, and the stress stimuli are contrived. However, measurement of cognitive stress level based upon raw EDA data of a wearable electronic device is difficult due to confounding environmental factors.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a computer-implemented method for detecting cognitive stress events using a wearable electronic device is disclosed. The computer-implemented method includes (i) segmenting an EDA sensor signal into rising and falling regions; (ii) segmenting rising regions and falling regions into a plurality of rise sub-regions and a plurality of fall sub-regions, respectively; (iii) fitting a transfer function to each rise sub-region of the plurality of rise sub-regions; (iv) fitting a decaying function to each fall sub-region of the plurality of fall sub-regions; (v) combining fit parameters to identify baseline rises; (vi) combining decaying fit parameters to identify baseline falls; and (vii) identifying or characterizing physiological events, based upon the baseline rises and the baseline falls, for detecting the cognitive stress events. The wearable electronic device includes at least one electrodermal activity (EDA) sensor and at least one contact area.

In another aspect, a wearable electronic device for detecting cognitive stress events is disclosed. The wearable electronic device includes at least one electrodermal activity (EDA) sensor, at least one contact area, at least one non-transitory memory storing instructions, and at least one processor communicatively coupled with the at least one non-transitory memory and configured to execute the instructions to perform operations including (i) segmenting an EDA sensor signal into rising and falling regions; (ii) segmenting rising regions and falling regions into a plurality of rise sub-regions and a plurality of fall sub-regions, respectively; (iii) fitting a transfer function to each rise sub-region of the plurality of rise sub-regions; (iv) fitting a decaying function to each fall sub-region of the plurality of fall sub-regions; (v) combining fit parameters to identify baseline rises; (vi) combining decaying fit parameters to identify baseline falls; and (vii) identifying or characterizing physiological events, based upon the baseline rises and the baseline falls, for detecting the cognitive stress events.

In yet another aspect, a non-transitory computer-readable media storing instructions thereon is disclosed. The instructions, when, executed by at least one processor of a wearable electronic or a computing device, cause the wearable electronic device or the computing device to detect cognitive stress events by performing operations including (i) segmenting an EDA sensor signal into rising and falling regions; (ii) segmenting rising regions and falling regions into a plurality of rise sub-regions and a plurality of fall sub-regions, respectively; (iii) fitting a transfer function to each rise sub-region of the plurality of rise sub-regions; (iv) fitting a decaying function to each fall sub-region of the plurality of fall sub-regions; (v) combining fit parameters to identify baseline rises; (vi) combining decaying fit parameters to identify baseline falls; and (vii) identifying or characterizing physiological events, based upon the baseline rises and the baseline falls, for detecting the cognitive stress events.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A illustrates an example wearable electronic device;

FIG. 1B illustrates an example block diagram of the wearable electronic device shown in FIG. 1A; and

FIG. 2 is a flow-chart of method operations for detecting cognitive stress events using the wearable electronic device shown in FIG. 1A or FIG. 1B.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

Some structural or method features may be shown in specific arrangements and/or orderings in the drawings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments, and, in some embodiments, it may not be included or may be combined with other features.

### DETAILED DESCRIPTION

Reference will now be made in detail to representative embodiments/aspects illustrated in the accompanying drawings. It should be understood that the following description is not intended to limit the embodiments to one preferred embodiment. On the contrary, it is intended to cover alternatives, modifications, and equivalents as can be included within the spirit and scope of the described embodiments as defined by the appended claims.

Embodiments described herein provide an apparatus and a method for real-time analysis of sensor data from one or more sensors included in a wearable electronic device. As described herein, the real-time analysis of sensor data is challenging due to artifacts introduced in the sensor data because of various noise sources and/or environmental factors including motion, humidity, and/or temperature, etc. Additionally, or alternatively, missing sensor data due to the environmental factors also makes real-time and accurate analysis of the sensor data a challenging task.

As described herein, EDA correlates with cognitive stress due to autonomic nervous system (ANS) activity. However, in practice, accessing the cognitive stress level from raw EDA data is typically only easy in lab-like environments where a subject person is unusually still and the stress stimuli are contrived for the cognitive stress measurement. For real life data, the problem is extremely difficult to solve when the cognitive stress is being measured based upon EDA using sensor data of an EDA sensor (or a galvanic skin response (GSR) sensor). The EDA sensor may be disposed within or on an external body of the wearable electronic device.

Cognitive stress is measured based upon certain features, for example, ANS evaporation peaks, of the EDA signal. However, these features are difficult to distinguish from noise in the signal, for example, due to motion artifacts. Additionally, even when certain morphological features are present in the EDA signal, they vary dramatically in size, shape and duration between individuals making typical signal processing methods ineffective in discovering morphological features. Finally, since stress events can last for relatively long periods of time (in excess of 15 minutes), typical approaches in deep learning relying on signal hysteresis become prohibitive due to the number of historical samples required to solve the problem. For all of these reasons, a novel baseline analysis technique (or baseline analysis algorithm) is required that can characterize morphology across different sizes, time, and shape scales to detect cognitive stress.

In an example embodiment, a real time cognitive stress is measured by combining raw input signals from several of EDA sensors, an accelerometer, a drive voltage sensor, a temperature sensor, and/or hardware flag anomalies into a new and corrected EDA signal. By way of a non-limiting example, the corrected EDA signal is generated in real time at the wearable electronic device because regular signal processing (or offline signal processing) is insufficient. The EDA signal is corrected using methods for real time deviant detection, gap interpolation across anomalies, and signal correction that can be applied to each of the sensor signals of interest. Upon generating or receiving the corrected real time signal, the baseline analysis algorithm may be performed by (i) segmenting the signal into regions of rising and falling baseline, ignoring small/local variations that are not true baseline variance; (ii) for each rise segment, performing a Gaussian segment analysis using extrema; (iii) for each region in the Gaussian segment analysis, fitting or using a nonlinear transfer function to characterize the amplitude and slope of that segment; (iv) combining fit parameters to form novel quantities identifying physiological baseline rises from those due to noise or environmental factors; (v) for each fall segment, attempting to fit (or fitting) a decaying function; and (vi) combining decaying fit parameters to identify value baseline falls due to physiological evaporation events, each of which is described in detail in the present disclosure. In the present disclosure, a sensor signal and a signal may be used interchangeably.

### Signal Segmentation

In an example embodiment, a signal is segmented into rising and falling regions. Due to noise and other environmental factors, there may be localized rises and falls that are not actual baseline changes. Accordingly, the smaller rises and falls are removed. Noise removal may be performed by a filtering technique that does not introduce a phase shift into the sensor signal. For example, the filtering technique used for noise removal may include one or more of wavelet filtering, double forward/reverse filtering, and total variance minimization. Additionally, or alternatively, any regions that are within a few seconds (or user provided time interval in seconds) of each other are merged together for signal segmentation. By way of a non-limiting example, the user provided time interval in seconds may be one second, two seconds, or five seconds, or any other time interval in seconds or milliseconds.

### Initial Rise/Fall Discovery

In an example embodiment, after the “varying” or different regions of the signal are discovered, the basic regions of rise/fall of the signal are detected using simple derivatives. If the derivatives of sufficient contiguous sets of samples are all positive, the region is constituted or identified as a rise of the sensor signal. Similarly, if the derivatives of sufficient contiguous sets of samples are all negative, the region is constituted or identified as a fall of the sensor signal.

Once the initial rise and fall regions are discovered, the discovered regions are refined to address real life environmental issues. For example, a long baseline rise is interrupted by smaller regions where the signal decreases. These smaller regions may be large enough to satisfy the criteria for minimum fall width. However, their presence interrupts the morphology of the larger rise, which is a focus of discovery of rise regions. As described herein, physiological characteristics of actual cognitive stress generally vary across scales in time, width and shape. As a result, typical pattern matching, wavelet, or other time-based feature extraction methods may not be efficient.

In an example embodiment, unnatural interruptions in rise and/or fall regions of the sensor signal are fixed by performing a series of steps in an order of jitter merging, priority unmixing, riffle merging, and variance filtering for rises. Jitter is defined as consistent oscillation between positive and negative derivatives, and jitter merging merges regions of neighboring rises or falls as long as there are no regions of the opposite kind in between them, and merging the regions will not exceed a jitter parameter threshold. In priority unmixing, two regions having overlapping edges, the region having a lower priority is discarded in favor of the region having a higher priority. When neighboring regions oscillate between rises and falls, a long rise interrupted by many smaller falls, or a long fall interrupted by many smaller rises, smaller interruptions are removed from the larger baseline in the riffle merging step. The rises that pass the jitter merging and the riffle merging, a total rise range must be large enough relative to the rest of the signal for the variance filtering step for rises; otherwise, the rises are considered as not belonging to the baseline's changes.

### Jitter Merging

Jitter is computed by counting the number of positive derivatives within a region of the sensor signal and comparing it to the number of negative derivatives. A ratio of the number of positive derivatives to the number of negative derivatives defines the jitter. When a signal is mostly increasing, with very few small deviations for signal decrease, the ratio of negative to positive derivatives will be extremely small, or the ration of positive to negative derivatives will be extremely high. Conversely, as the number of positive vs. negative derivatives reaches parity, the ratio approaches 1.

If neighboring regions with short gaps between them (for example, a few seconds) can be merged together without exceeding a jitter threshold, then such neighboring regions are combined; Otherwise, such neighboring regions are discarded. Before comparing the ratio of positive to negative derivatives, a filter is first applied so that only derivatives having absolute value larger than the threshold value are included or considered for the next step.

### Priority Unmixing

The priority unmixing step is relatively simple but necessary. As described herein, if two regions overlap, the lower priority region is discarded in favor of the higher priority region. For long rises, the rise is the higher priority region, with smaller falls having low priority region and being discarded. Similarly, for long falls, the opposite is true.

### Riffle Merging

Riffling occurs when the varying regions in the signal keep switching back and forth between rises and falls. For long changes in the baseline, the higher priority regions are kept and the lower priority regions are merged out. For example, a long rise with small, intermittent falls may be merged into a single long rise according to a method described herein. The method described herein for the rise may also be applied for the fall. If a rise and fall are within a few seconds of each other, but the falls are shorter than a physiological threshold, then the intermittent falls are replaced with a longer, continuous rise. For long falls, if the rises are close to the falls and shorter than the physiological threshold for falls, the intermittent rises are placed with longer, continuous falls. Finally, a jitter filter is applied by removing regions whose jitter value exceeds the riffle merge threshold.

### Variance Filtering

As described herein, variance filtering is a final step for rises only, which ensures that a rise has sufficient increase in absolute signal magnitude to constitute an actual baseline shift. A tolerance is calculated relative to the standard deviation of the signal. For EDA and stress, valid baseline rises increase the signal magnitude by several times the standard deviation of the signal. Any rises below this threshold, for example, the standard deviation of the signal, are discarded.

### Gaussian Segment Analysis (Rises)

In an example embodiment, for each identified valid rise a Gaussian segment analysis is performed to find one or more sub-segments that should be fit with transfer functions, as described in detail below. The Gaussian segment analysis is performed by applying a median filter, computing the derivative of the signal and convolve a Gaussian window. Peaks and troughs in the Gaussian-convolved signal are discovered, and extrema from the signal are used as critical points dividing the signal into Gaussian segments.

### Transfer Function Fits (Rises)

In an example embodiment, once the Gaussian segments of the signal are identified, a transfer function f is fitted to each segment, for example, as y₀+Af(α(x−x₀)). Finally, it is ensured that the objective function used for the optimization takes the derivative of the signal into account. By way of a non-limiting example, for transfer function fits of this type, the fit may have symmetric errors in certain cases so that the fit becomes a straight line with errors canceling on either side of the point where the fit and the original signal meet.

### Decaying Function Fits (Falls)

In an example embodiment, depending on the signal, one of several decaying functions may best describe the fall. The decaying function is accordingly selected that is the closest in morphology to the natural decays in the physiological baseline of the signal. By way of a non-limiting example, decaying function may include, but is not limited to, decaying exponential, power law, decaying sections of any standard statistical distribution, or linear/non-linear combinations and compositions of these functions. A non-linear fit is performed for each identified fall segment and various parameters are extracted for interpretation or characterization, as discussed herein.

### Rise Characterization

In an example embodiment, once the transfer functions have been fit to each rise segment, amplitude and slope fit parameters of the transfer function are extracted and used to create a vector describing the rise. As described herein, the physiological baseline rises in EDA for each individual will be different across scales in time, amplitude and morphology. By performing a Gaussian-based segmentation, various scaling issues may be addressed or solved. For longer periods of time, more Gaussian segments may be generated. For larger amplitude increases, larger individual segments, or more segments, may be generated depending on the morphology of the signal. Additionally, for morphological differences in scaling, a different transfer function may be selected even at the individual segment level.

By way of a non-limiting example, in an example embodiment, the amplitude and slope fit parameters from each segment are compared to the segment, and the differences between idealized fit of the segment and the segment itself are quantified for detection of cognitive stress from EDA because physiological signals tend to have the idealized shapes of transfer functions in real life. When a baseline shift is due to physiology, the signal closely represents a transfer function. When a baseline shift is due to noise, motion, or other environmental factors, the signal may approximate a transfer function somewhat, but the difference between ideal fit and baseline signal is generally greater than in the physiological case, which can be more easily detectable.

### Fall Characterization

In an example embodiment, the decaying fits are characterized by the parameters of the non-linear fit. For decays, the most important parameter may be the characteristic decay time. Whatever physiological phenomenon is being measured, it usually has time bounds associated with real, physiological responses in the body. As such, once falls are identified and had decaying functions fit to them, physiological falls are identified by their decay times falling within the region of acceptable decay times associated with the phenomenon. For cognitive stress, the ANS evaporation events happen at a microscale and a macro scale. To identify stress events, the macro scale evaporation events are the most important events occurring over a period of 5 to 20 minutes, depending on the severity of the stress event.

By combining the rise and fall characterizations, regions of actual cognitive stress may be identified that are not due to environment or noise. Further, the identified regions of actual cognitive stress may be classified as low, medium, or high stress regions using the decay constants, and the number and slope of the rise fits.

### Cognitive Stress Region Detection

In an example embodiment, upon identifying the set of rises and falls, stress regions may be detected based upon maintaining a stack of rises in which if multiple rises occur in succession without a fall, the multiple rises are treated as a continuation of the same rise event. When a fall is detected or encountered, an amount of time that has lapsed since the first rise is checked. If the detected amount of time is between, for example, 2 and 15 minutes, an intervening region between the first rise and the detected fall is identified or flagged as a stress event. Whether the stress event is a low-level, medium-level, or high-level stress event is determined based upon the characteristic decay time. Further, the total baseline changes due to the one or more rises to the total baseline changes from the one or more decay events are compared to ensure that they match. However, because longer term trends in the baseline may still appear due to temperature and humidity changes in the environment, multiple sequential rises and falls on the stack are observed or used to ensure that they are paired up appropriately.

By way of an example, when there are multiple stress events over time, their relative baseline changes begin to form a distribution that is customized to the individual while allowing greater confidence in identifying future stress event probabilities, and classifying any particular event as being high-level, medium-level or low-level stress event. Also, because cognitive stress levels are subjective, and a person is unlikely to characterize every cognitive stress event as a high-level stress event on a given day, the cutoff on the distribution of events may be chosen such that there is a maximum number of high-level stress events allowed on any given single day.

Once distributions of stress events are formed based on strength and duration, long-term trends in cognitive stress may be identified or determined by looking at the morphology of the event distributions across time duration and baseline increases. Various distribution morphologies correlate with the cognitive stress resilience of an individual. Additionally, or alternatively, distributions of stress events may also be used to track interventions such as mindfulness exercises, other things.

Although EDA signals correlate well with cognitive stress in lab environments, signals obtained in free living are extremely difficult to analyze and correlate with actual stress due to massive differences in the duration, range, and morphology of the baseline changes for each individual during cognitive stress events. Because these issues exist in all three of these categories, normal signal processing methods and simple time series features are unable to distinguish between actual stress events and noise. To solve this problem, a baseline analysis, as described herein, is conducted for tracking and characterizing changes in the EDA baseline that allow the accurate detection of stress in real time, and in free living environments. As described herein, the method described herein offers a significant benefit that the method can be used despite noise and environmental factors, and can handle the scaling issues across duration, range and morphology of the EDA signal.

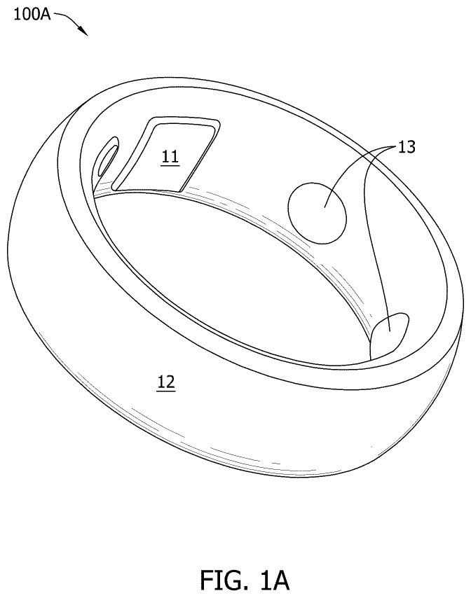

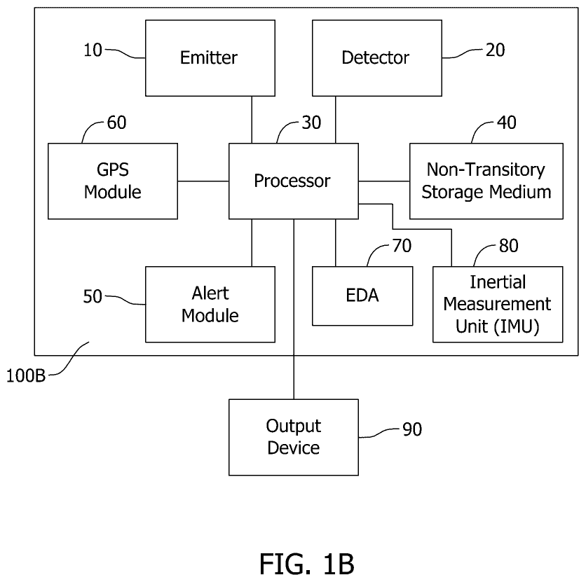

FIG. 1A illustrates an example wearable electronic device 100A. The wearable electronic device 100 may be worn by a user or secured to the user. By way of an example, the wearable electronic device may include, but is not limited to, a wearable computer, a wearable watch, a wearable communication device, a wearable ring, a wearable media player, a wearable health monitoring device, and the like. In one example, the wearable electronic device 100A may be a wearable electronic device including multiple functionalities such as time keeping, health monitoring, sports monitoring, medical monitoring, stress monitoring, communications, navigation, computing operations, monitoring a user's physiological and/or biological signals and providing health-related information based on those signals, and/or communicating with other electronic devices or services in a wired or wireless manner. The wearable electronic device 100A may also provide alerts to the user, which may include one or more of audio, haptic, visual and/or other sensory output. The wearable electronic device 100A may also display data on a display device of the wearable electronic device 100A and acquire sensor data form one or more sensors positioned at or within the wearable electronic device 100A.

The wearable electronic device 100A includes a device body 11 including a housing that carries, encloses, and supports both externally and internally various components (including, for example, integrated circuit chips and other circuitry) to provide computing and functional operations for the wearable electronic device 100A. The components may be disposed on the outside of the housing, partially within the housing, through the housing, completely inside the housing, and the like. The housing may, for example, include a cavity for retaining components internally, holes or windows for providing access to internal components, and various features for attaching other components. The housing may also be configured to form a water-resistant or water-proof enclosure for the device body 11. For example, the housing may be formed from as a single unitary body and the openings in the unitary body may be configured to cooperate with other components to form a water-resistant or water-proof barrier. By way of a non-limiting example, the device body 11 may include components such as, but not limited to, processing units, memory, display, sensors, biosensors, speakers, microphones, haptic actuators, batteries, and so on. The wearable electronic device 100A may also include a band 12 or strap or other means for attaching to a user. In the example wearable electronic device 100A, the band 12 has an annular shape with an aperture to receive a finger of a subject user.

Additionally, the device body 11 may have one or more contact areas 13 for cognitive stress measurement for the user. By way of an example, the one or more contact areas may be provided as one or more buttons on the sides of the device body 11. Additionally, or alternatively, EDA sensors having metallic or conductive areas may be positioned on the bottom of the device body 11 such that the EDA sensors come into contact with skin of the subject user. These contact areas serve as electrodes for measuring electrical conductance of the skin through a combination of high-frequency and low-frequency measurements.

FIG. 1B illustrates components of the wearable electronic device 100B according to an example of this disclosure. As shown in FIG. 1B, the wearable electronic device 100B includes an emitter 10 and detector 20, which are coupled to a processor 30. The processor 30 is coupled to a non-transitory storage medium 40. The wearable electronic device 100B is coupled to an output device 90. The wearable electronic device 100B is the wearable electronic device 100A shown in FIG. 1A.

The emitter 10 delivers light to a tissue and the detector 20 collects the optically attenuated signal that is backscattered from the tissue. In at least one example, the emitter 10 can be configured to emit at least three separate wavelengths of light. In another example, the emitter 10 may be configured to emit at least three separate bands or ranges of wavelengths. In at least one example, the emitter 10 may include one or more light emitting diodes (LEDs). The emitter 10 may also include a light filter. The emitter 10 may include a low-powered laser, LED, or a quasi-monochromatic light source, or any combination thereof. The emitter may emit light ranging from infrared to ultraviolet light. As indicated above, the present disclosure uses Near-Infrared Spectroscopy (NIRS) as a primary example and the other types of light can be implemented in other examples and the description as it relates to NIRS does not limit the present disclosure in any way to prevent the use of the other wavelengths of light.

The data generated by the detector 20 may be processed by the processor 30, such as a computer processor, according to instructions stored in the non-transitory storage medium 40 coupled to the processor. The processed data can be communicated to the output device 90 for storage or display to a user. The displayed processed data may be manipulated by the user using control buttons or touch screen controls on the output device 90 or on the device body 11.

The wearable electronic device 100B may include an alert module 50 configured to generate an alert. The processor 30 may send the alert to the output device 90 or the alert module 50 may send the alert directly to the output device 90. In at least one example, the wearable electronic device 100B may be configured so that the processor 30 is configured to send an alert to the output device 90 without the device including an alert module 50.

The alert may provide notice to a user, via a speaker or display on the output device 90, of a change in biological indicator conditions or other parameter being monitored by the wearable electronic device 100B, or the alert may be used to provide an updated biological indicator level to a user. In at least one example, the alert may be manifested as an auditory signal, a visual signal, a vibratory signal, or combinations thereof. In at least one example, an alert may be sent by the processor 30 when a predetermined biological indicator event occurs during a physical activity.

In at least one example, the wearable electronic device 100B may include a Global Positioning System (GPS) module 60 configured to determine geographic position and tagging the biological indicator data with location-specific information. The wearable electronic device 100B may also include an EDA sensor 70 and an IMU 80. The IMU 80 may be used to measure, for example, gait performance of a runner or pedal kinematics of a cyclist, as well as physiological parameters of a user during a physical activity. The EDA sensor 70 and IMU 80 may also serve as independent sensors configured to independently measure parameters of physiological threshold. The EDA sensor 70 and IMU 80 may also be used in further algorithms to process or filter the sensor signals data. The wearable electronic device 100B may also include other types of sensors, for example, a thermistor, and/or a heart rate sensor.

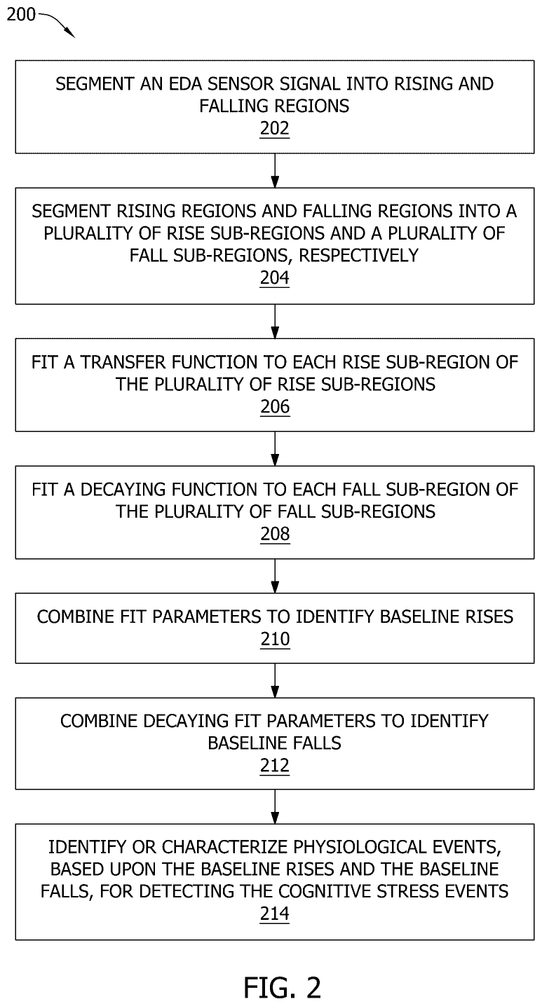

FIG. 2 is a flow-chart 200 of method operations for detecting cognitive stress events using a wearable electronic device such as the wearable electronic device 100A shown in FIG. 1A or 100B shown in FIG. 1B, respectively. The wearable electronic device includes at least one EDA sensor and at least one contact area for a user of the wearable electronic device to place one or more fingers or a portion of palm area of the user. The method operations include segmenting 202 an EDA sensor signal into rising and falling regions. The EDA sensor signal segmentation is performed using a filtering technique such as, wavelet filtering, double forward/reverse filtering, and total variance minimization. Because segmenting 202 the EDA sensor signal into the rising and falling regions is described in detail earlier, those details are not repeated here for the sake of brevity.

The method operations include segmenting 204 rising regions and falling regions into a plurality of rise sub-regions and a plurality of fall sub-regions, respectively. As described herein, unnatural interruptions occur in rise and/or fall regions of the sensor signal. The interruptions thus cause a plurality of sub-regions for the rising regions and the falling regions. Accordingly, a plurality of rise sub-regions and a plurality of fall sub-regions are identified through segmenting 204. Further, as described herein, the plurality of rise sub-regions and the plurality of fall sub-regions are processed using jitter merging, priority unmixing, riffle merging, and variance filtering for rises, and boundaries of the plurality of rise sub-regions are identified using Gaussian kernel convolution, amplitudes, slopes, and their extrema.

The method operations include fitting 206 a transfer function to each rise sub-region of the plurality of rise sub-regions and fitting 208 a decaying function to each fall sub-region of the plurality of fall sub-regions. The transfer function for each rise sub-region of the plurality of sub-regions may include any of a hyperbolic function, a linear function, a rectified linear function, and a sigmoidal or Gaussian function. The decaying function for each fall sub-region of the plurality fall sub-regions may include any of a decaying exponential function, Boltzmann distribution, power law, or blackbody radiation function.

The method operations include combining 210 fit parameters to identify baseline rises and combining 212 decaying fit parameters to identify baseline falls. The fit parameters are used to characterize one or more of the morphology, range, and duration of a rise event. Further, the fit parameters are used to characterize whether a rise is physiological or due to noise or environmental factors. Similarly, the decaying fit parameters are used to characterize one or more of the morphology, range, and duration of a fall event, and to characterize whether a fall is physiological or due to the noise or environmental factors. As described herein, the rise characterization marks a start of a cognitive stress event of events, and the fall characterization marks an end of the cognitive stress event of a plurality of events.

The method operations include identifying or characterizing 214 physiological events, based upon the baseline rises and the baseline falls, for detecting the cognitive stress events. The events are labeled as physiological based on a time difference between one or more rises and corresponding one or more falls, as described herein. Further, the events may be labeled as low-level, medium-level, or high-level cognitive stress events based at least in part upon a time difference between one or more rises and one or more falls and based at least in part upon characterizations of the rises and falls. The characterizations form custom distributions for defining typical baseline increase, duration, and morphology across the fit parameters of both rises and falls.

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

One may appreciate that although many embodiments are disclosed herein, that the operations and steps presented with respect to methods and techniques described herein are meant as exemplary and accordingly are not exhaustive. One may further appreciate that alternate step order or fewer or additional operations may be required or desired for particular embodiments.

Although the disclosure herein is described in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of some embodiments, whether or not such embodiments are described and whether or not such features are presented as being a part of a described embodiment. Thus, the breadth and scope of the present description should not be limited by any of the exemplary embodiments described herein but is instead defined by the claims herein presented.

What is claimed is:

1. A computer-implemented method for detecting cognitive stress events using a wearable electronic device including at least one electrodermal activity (EDA) sensor and at least one contact area, the computer-implemented method comprising:
segmenting an EDA sensor signal into rising and falling regions;
segmenting rising regions and falling regions into a plurality of rise sub-regions and a plurality of fall sub-regions, respectively;
fitting a transfer function to each rise sub-region of the plurality of rise sub-regions;
fitting a decaying function to each fall sub-region of the plurality of fall sub-regions;
combining fit parameters to identify baseline rises;
combining decaying fit parameters to identify baseline falls; and
identifying or characterizing physiological events, based upon the baseline rises and the baseline falls, for detecting the cognitive stress events.

2. The computer-implemented method of claim 1, wherein segmenting the EDA sensor signal is performed using total variance minimization.

3. The computer-implemented method of claim 1, further comprising identifying boundaries of the plurality of rise sub-regions using Gaussian kernel convolution, amplitudes, slopes, and their extrema.

4. The computer-implemented method of claim 1, wherein fitting the transfer function to each rise sub-region of the plurality of rise sub-regions comprises fitting a hyperbolic function.

5. The computer-implemented method of claim 1, wherein fitting the transfer function to each rise sub-region of the plurality of rise sub-regions comprises fitting a linear or rectified linear function.

6. The computer-implemented method of claim 1, wherein fitting the transfer function to each rise sub-region of the plurality of rise sub-regions comprises fitting a sigmoidal or Gaussian function.

7. The computer-implemented method of claim 1, wherein fitting the decaying function to each fall sub-region of the plurality of fall sub-regions comprises fitting a decaying exponential function, Boltzmann distribution, power law, or blackbody radiation function.

8. The computer-implemented method of claim 1, wherein the fit parameters are used to characterize one or more of the morphology, range, and duration of a rise event.

9. The computer-implemented method of claim 1, wherein the decaying fit parameters are used to characterize one or more of the morphology, range, and duration of a fall event.

10. The computer-implemented method of claim 1, wherein the fit parameters are used to characterize whether a rise is physiological or due to noise or environmental factors, and wherein the decaying fit parameters are used to characterize whether a fall is physiological or due to the noise or environmental factors.

11. The computer-implemented method of claim 10, wherein the rise characterization marks a start of a cognitive stress event of events, and wherein the fall characterization marks an end of the cognitive stress event of the events.

12. The computer-implemented method of claim 11, wherein the events are labeled as physiological based on a time difference between one or more rises and corresponding one or more falls.

13. The computer-implemented method of claim 11, wherein the events are labeled as low-level, medium-level, or high-level cognitive stress events based at least in part upon a time difference between one or more rises and one or more falls and based at least in part upon characterizations of the rises and falls.

14. The computer-implemented method of claim 13, wherein the characterizations form custom distributions for defining typical baseline increase, duration, and morphology across the fit parameters of both rises and falls.

15. The computer-implemented method of claim 14, wherein the low-level, medium-level, or high-level cognitive stress events are personalized using the custom distributions such that a maximum number of high-level stress events is possible in a single day.

16. A wearable electronic device for detecting cognitive stress events, the wearable electronic device comprising:
at least one electrodermal activity (EDA) sensor;
at least one contact area,
at least one memory storing instructions; and
at least one processor communicatively coupled with the at least one memory and configured to perform operations comprising:
segmenting an EDA sensor signal into rising and falling regions;
segmenting rising regions and falling regions into a plurality of rise sub-regions and a plurality of fall sub-regions, respectively;
fitting a transfer function to each rise sub-region of the plurality of rise sub-regions;
fitting a decaying function to each fall sub-region of the plurality of fall sub-regions;
combining fit parameters to identify baseline rises;
combining decaying fit parameters to identify baseline falls; and
identifying or characterizing physiological events, based upon the baseline rises and the baseline falls, for detecting the cognitive stress events.

17. The wearable electronic device of claim 16, wherein segmenting the EDA sensor signal is performed using total variance minimization, and wherein the operations further comprising identifying boundaries of the plurality of rise sub-regions using Gaussian kernel convolution, amplitudes, slopes, and their extrema.

18. The wearable electronic device of claim 16, wherein fitting the transfer function to each rise sub-region of the plurality of rise sub-regions comprises fitting a hyperbolic function, a linear function, rectified linear function, a sigmoidal function or Gaussian function, and wherein fitting the decaying function to each fall sub-region of the plurality of fall sub-regions comprises fitting a decaying exponential function, Boltzmann distribution, power law, or blackbody radiation function.

19. The wearable electronic device of claim 16, wherein:
the fit parameters are used to characterize one or more of the morphology, range, and duration of a rise event;
the decaying fit parameters are used to characterize one or more of the morphology, range, and duration of a fall event;
the fit parameters are used to characterize whether the rise event is physiological or due to noise or environmental factors; and
the decaying fit parameters are used to characterize whether the fall event is physiological or due to the noise or environmental factors.

20. A non-transitory computer readable media storing instructions thereon, which, when executed by at least one processor of a wearable electronic device comprising at least one electrodermal activity (EDA) sensor and at least one contact area, cause the wearable electronic device to detect cognitive stress events by performing operations comprising:
segmenting an EDA sensor signal into rising and falling regions;
segmenting rising regions and falling regions into a plurality of rise sub-regions and a plurality of fall sub-regions, respectively;
fitting a transfer function to each rise sub-region of the plurality of rise sub-regions;
fitting a decaying function to each fall sub-region of the plurality of fall sub-regions;
combining fit parameters to identify baseline rises; and
combining decaying fit parameters to identify baseline falls; and
identifying or characterizing physiological events, based upon the baseline rises and the baseline falls, for detecting the cognitive stress events.
